(19) United States
(12) Patent Application Publication
(10) Pub. No.: US 2017/0319148 A1
(43) Pub. Date: Nov. 9, 2017

(54) SMART MIRROR AND PLATFORM

(71) Applicant: Mimitec Limited, London (GB)

(72) Inventors: Tamer Mohamed Mounir Shahin, London (GB); Omran Alsayed Alhashmi, Shahamah (AE); Erfan Alsayed Alhashmi, Shahamah (AE)

(21) Appl. No.: 15/146,271

(22) Filed: May 4, 2016

(51) Int. Cl. A61B 5/00; H04N 5/33; H04N 5/247; G06F 3/048; A61B 5/16; A61B 5/05; A61B 5/01; H04N 13/02; A61B 1/00

(52) U.S. Cl. CPC A61B 5/743; H04N 13/0203; H04N 5/33; H04N 5/247; G06F 3/048; A61B 1/00163; A61B 5/4884; A61B 5/165; A61B 5/0507; A61B 5/015; A61B 5/7465

(57) ABSTRACT

A system for determining biological information of a subject includes a surface configured to display an image of a subject positioned in front of the surface, a sensor configured to identify data of the subject, and an analysis and control unit configured to analyse the data and determine biological information of the subject based on the data.

### BACKGROUND OF THE INVENTION

### 1. Field of the Invention

The invention relates to a system for determining biological information of a subject.

### 2. Description of the Related Art

Finding improvements in health care, as opposed to sick care, is an on-going global challenge. It has hitherto been notoriously difficult to achieve real and sustained engagement and behavioural change to improve a person's health.

Contemporary research has shown that seven out of ten deaths are due to chronic disease and around 86% of the trillion dollar health care budget in the US is spent on such diseases. Yet chronic diseases are also the most preventable and manageable diseases through actionable and accessible data which influences positive behaviour. For example, roughly half of patients with diabetes stop treatment within the first year. This directly leads to a large amount of waste of resources. The USA alone loses $564b. or 87% of potential revenue each year due to non-adherence to medication. Research has shown that there is a 27% increase in diabetes medication adherence (and revenue) from simple reminders being provided to patients.

Several technologies exist to be able to monitor one's activities or vital signs however the problem with existing technologies are that they are hard work and lack continuity or connectivity to create a real and meaningful understanding of one's health.

There is therefore a need to provide a simple, reliable and meaningful system to enable a subject to gain an understanding of their own health.

### SUMMARY OF THE INVENTION

According to a first aspect of the invention there may be provided a system for determining biological information of a subject, comprising: a surface configured to display an image of a subject positioned in front of the surface; a sensor configured to identify data of the subject; an analysis and control unit configured to analyse the data and determine biological information of the subject based on the data.

Preferably the surface comprises a mirrored surface. The surface may comprise a display. The system may comprise a camera configured to output to the display an image retrieved by the camera. Preferably the mirrored surface may be a dielectric mirror surface arranged adjacent to a display.

In some embodiments the sensor may be a thermal infrared “IR” camera. The analysis unit may be configured to determine skin temperature based on data from the thermal IR camera.

In some embodiments the sensor may be a microwave pulse sensor configured to omit a signal and receive reflected signals, the reflected signals signal including at least part of the omitted signal reflected off the subject. The biological information may be heart rate. The biological information may be respiratory rate.

The system may further comprise an ambient temperature sensor. The analysis unit may be configured to calibrate measurements based on the data.

The sensor may be a microphone. The biological information may be voice characteristics. The analysis unit may be further configured to identify instructions based upon recognising voice in the data.

The sensor may be a camera. The biological information may be height. The analysis unit may be configured to determine body movement based on the data. The analysis unit may be configured to determine gestures based on the data.

The sensor may be a 3D depth sensor apparatus.

In some embodiments the system may further comprise one or more force sensors. The one or more force sensors may be configured to communicate wirelessly with the analysis unit. The biological information may be weight. The system further comprises a camera and the analysis unit may be configured to determine BMI based on the data and an image retrieved from the camera. The biological information may be a measure of balance of the subject. The biological information may be a posture of the subject.

In certain embodiments the biological information may be any one or more of mood, age, gender and/or stress.

Further the system comprises a plurality of different sensors and the biological information may be determined based on a combination of the data retrieved from each of the sensors.

The analysis unit may be configured to output the biological information to the display.

The analysis unit may be configured to retrieve subject medical information and output a reminder of a task based on the medical information to the display.

Optionally the system further comprises a digital platform. The digital platform may be configured to receive biological information from the analysis unit and store the information in a database. The digital platform may be configured to receive the data and wherein the analysis unit is comprised in the platform. The platform may be separate from the one or more sensors and separate from the display. The system may comprise a series of application programing interfaces “APIs”, each API providing access for third party components to the data or the biological information or both. The system may comprise a series of APIs, each API providing access for the digital platform to third party sensors.

The system may comprise an algorithm library comprising a series of algorithms for the analysis unit to use when determining biological information from the data. The system may comprise a remote module comprising a series of algorithms for the analysis unit to use when determining biological information from the data. The system may comprise a module which enables predictive analytics of future conditions of the subject based on the biological information. The system may comprise a module which enables predictive analytics of future conditions of the subject based on a detected change in biological information. The system may comprise a module comprising machine learning components.

In a further aspect, a method in a system comprising a mirrored surface may be provided, the system comprising a display and one or more sensors configured to identify data of a subject positioned in front of the mirrored surface, the method comprising: retrieving data from the one or more sensors; identifying a biological information of the subject based on the data; and, outputting the biological information to the display.

In a further aspect, a method may be provided in a digital platform in communication with a system comprising a mirrored surface and one or more sensors; the method comprising: receiving data of a subject from the one or more sensors; and analysing the data and determining biological information of the subject based on the data.

In a further aspect, a method may be provided in a digital platform in communication with a system comprising a mirrored surface, one or more sensors, and an analysis unit; the method comprising: receiving biological information from the analysis unit; and storing the biological information in a database.

In a further aspect, a computer readable medium may be provided comprising instructions which when executed by a computer cause the computer to: retrieve data from one or more sensors; identify biological information of a subject subject positioned in front of a mirrored surface adjacent to a display based on the data; and, outputting the biological information to the display.

### BRIEF DESCRIPTION OF THE DRAWINGS

Examples of systems and methods in accordance with the invention will now be described with reference to the accompanying drawings, in which:

FIG. 1 shows an image of an exemplary mirrored display according to the present invention;

FIG. 2 shows an exemplary high-level architecture according to the present invention;

FIG. 3 shows an exemplary architecture of a platform system according to the present invention; and,

FIG. 4, which is composed of FIGS. 4a-d as designated in FIG. 4, shows an exemplary overview of a system within which the platform may be configured according to the present invention.

### DETAILED DESCRIPTION

Proposed is a patient-centric solution promoting behavioural change through passive data collection and predictive analysis resulting in emotionally intelligent non-invasive diagnostics. It is proposed to provide a system which is able to measure biological information when a subject is positioned in front of a mirror. Preferably the measurement is passive. By combining passive data collection with a daily activity such as standing in front of a mirror, the system is able to overcome the problem of a lack of engagement with measurement technology.

As will become clear from the below, embodiments of the invention provide for seamless preventative care. In certain examples, the system is able to provide the following benefits: the system is able to provide patient empowerment through a smart user experience; the system is able to enable better care with emotionally intelligent recommendations of care and communications with a care provider; the system is able to reduce costs through active engagement, compliance, and disease prevention; the system is able to provide secure real-time data-driven innovation and support clinical trials and optimisation treatment protocols; and, additionally, the system is able to provide remote patient monitoring. Further, an increasingly aging population at risk of developing code-morbidities may be aided through the ability of the system to monitor patients remotely.

A specific example of the present invention will now be described. It will be understood that this is a specific, non-limiting example only. Anecdotally, subjects want help to get fit and manage their lifestyles to stay healthy and feel good but they are not technology savvy, cannot often be bothered with charging wearable technology, and often do not have the time to log into apps or upload data to the cloud in the end to get meaningless information. The example described overcomes these issues.

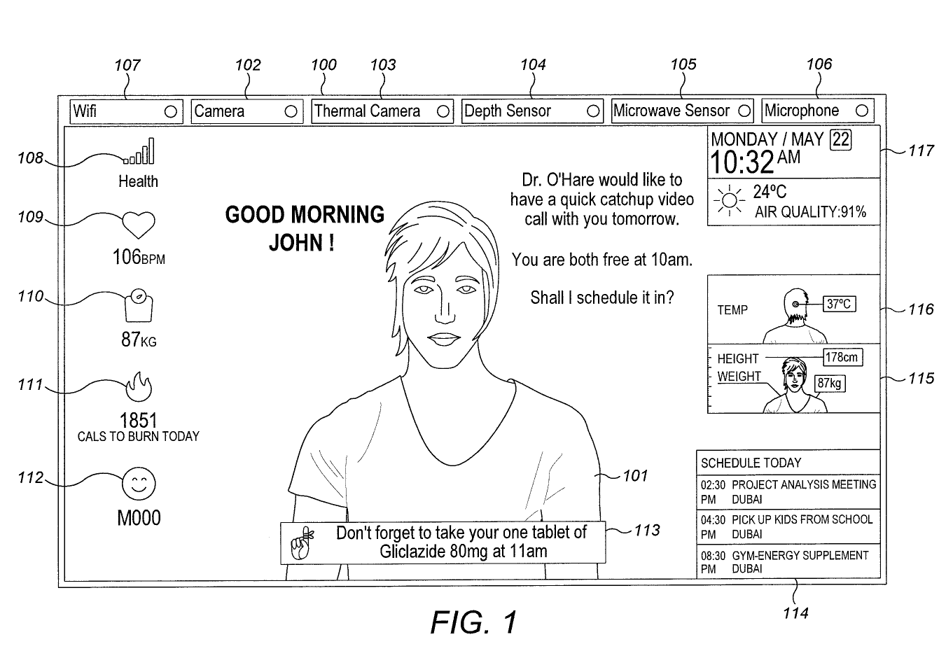

FIG. 1 illustrates a view of a mirror 100 of an example system from the perspective of a subject 101. Subject 101 is illustrated in front of the mirror 100 with his image being reflected back at him. The image of the subject 101 is provided with an overlaid graphical user interface displayed on a screen. Housed within the unit 100 is an array of sensors. Each sensor combines to identify, through sensing and tracking, aspects of the subject 101. For example, sensing and tracking of body movement, hand and finger gestures, posture, height, identification of age and gender, facial expressions and mood. Based on the sensors included in the housing 100 such as a camera 102, thermal camera 103, depth sensor 104, microwave sensor 105 and microphone 106, the system is able to detect biological information of the subject 101 such as heart rate, body temperature, height, motion and posture, moving stress levels, breathing, gender, age and facial recognition. Through a communication module 107 illustrated here as a Wi-Fi module 107, data can also be detected from other connected devices.

As shown in FIG. 1, a computer and display combine to show results of the detection to the subject. The system is also able to store or send the data to a remote unit. As mentioned above, the computer and display combine to provide an overlay to the user. The user may be notified of for example, a visual indication of their health 101. Through detection by the sensors, the system may be able to determine heart rate 109 and display this to the user. Weight 110 may also be presented.

A computer may also be able to display other data useful to the user such as a number of calories 111 one needs to burn in order to reach a particular goal. An indication of the mood 112 of the user could also be detected and presented.

The overlay may also show programmed messages or reminders or data retrieved from remote sensors. For example illustrated in FIG. 1 is a reminder 113 for the subject to take their medication at a particular time. Also illustrated is a retrieval of the subject's calendar for the day 114, an indication of their vital statistics 115, their body temperature 116 and an indication of the current time 117.

A number of further uses of the system are also contemplated. For example, by detecting movement of the user, and preferably over a particular time period, the system may be able to determine a period of time in which the user has brushed their teeth and accordingly, present a warning to the user if a sufficient time has not elapsed for the teeth to be sufficiently cleaned.

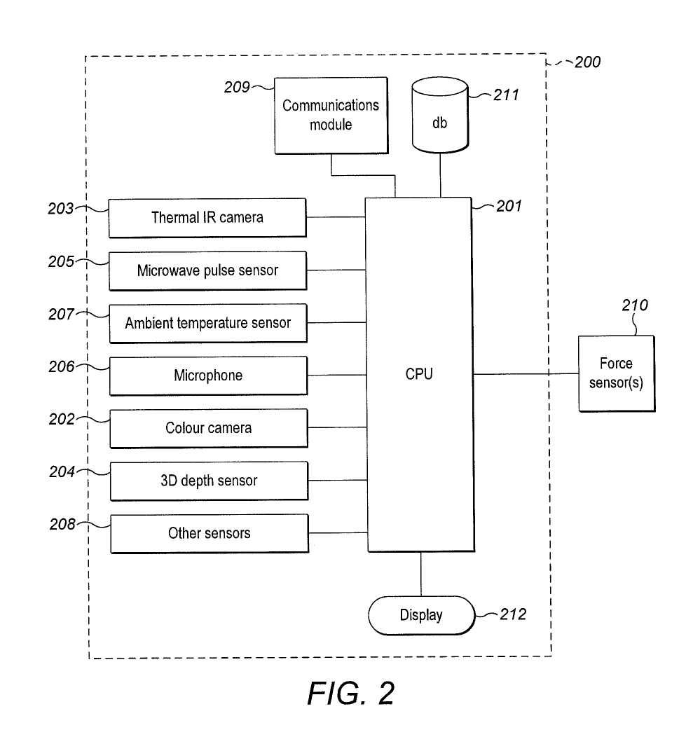

FIG. 2 illustrates a high-level block diagram of an example system comprising a mirror. Throughout the present description the terms system, housing, unit and mirror may be used interchangeably to represent the combination of components which together make up the unit and screen which enable the user to see representation of themselves together with an array of vital statistics.

The unit 200 is illustrated showing each component. To control and analyse the data a CPU 201 is provided. Although it is described that the control may be through a CPU 201, it will be understood that of course any reasonably programmed computer may be used. For example, a microcontroller, computer, processor and memory means or other appropriate component. It will be understood what is key to the functionality of the component, that is, to control the functions of the server, communicate with each component and analyse the data to present an output accordingly.

In communication with the CPU 201 is a series of sensors. Each sensor will now be described in turn. It will be understood that any combination of sensors may be used in order to detect and determine specific functionality or biological information of the subject.

A camera 202 may be provided within the housing in order to record images of the subject. While recording images of the subject, subsequent remote diagnosis may be performed. Further, through video analysis by the CPU 201 or remotely, biological information of the user can be determined. An example could be by looking at heatmap of face temperature to establish skin temperature. Also, one of the ways in which the system can used, in addition to the microwave sensor, to measure heart rate through video analysis. Also the system can identify changes in facial symmetry to detect possible onset of stroke as well as potentially in the future look at retina to detect risk of diabetes. Preferably the camera is colour and may be an RGB high definition video capturing camera at greater than or equal to 30 frames per second. To enable sufficient resolution to be captured a camera will preferably have greater than or equal to 12 megapixels.

A thermal infrared (IR) camera 203 may also be provided. Through use of an IR camera 203, the system is able to measure skin temperature or otherwise the temperature of the subject in front of the mirror. The thermal IR camera 203 may be a microbolometer. A microbolometer is an uncooled thermal sensor. When infrared radiation strikes a detector material an electrical resistance is changed. The resistance change is measured and processed into temperatures which can be used to create an image.

A 3D depth sensor module 204 may also be provided which may be a combination of a depth camera and infrared emitters. The depth sensor may be used to track body movement and other information from a subject. Typical depth sensors that may be used are well known in the art.

A microwave pulse sensor 205 may also be provided in the housing. In this example, microwaves reflect off the surface of the subject and the signal changes slightly in accordance with movements of the subject's heart and lungs. From these changes biological information such as heart rates and respiratory rates can be measured.

A microphone 206 may also be provided in the housing for voice recognition. The voice recognition may be used to detect ailments in the subject or to receive instructions for interacting with the user interface provided by the CPU 201. An example microphone is a 4-microphone array and 24 digital converter. The audio format may be a 16 kHz, 24 bit mono pulse code modulation.

In each case, the CPU 201 may build up a historical pattern of the user data and monitor changes in order to determine ailments. Alternatively, the CPU may be programmed to determine ailments from a combination of sensors.

In order to calibrate the sensors and provide error checking, an ambient temperature sensor 207 may be provided to enable comparison with the thermal IR sensor or other sensors. For example, the ambient temperature sensor 207 may be used to compare the skin temperature and current environment to make for any necessary corrections.

Other sensors 208 may be provided.

Additionally, fingerprints could be used for identification or a finger prick could be used to measure blood glucose.

In order to communicate remotely or to communication with other sensors external to the housing, a communications module 209 may be provided. It will be understood of course that any communications technologies may be used such as Wi-Fi, Bluetooth, RFID and wired communications. However, wireless is preferable.

External to the housing may optionally be provided a force sensor 210 which is able to determine weight and balance. For example, the force sensor 210 may be positioned on a floor on which the subject is to stand when facing the mirror. The sensor 210 may communicate wirelessly with the CPU 201 through communication modules 209.

The optional force sensor 210 may allow for weight to be determined along with height for BMI measurement and calories to burn etc. Additionally, balance sensors may be able to determine posture and potential weaknesses on one side of the body.

The CPU 201 may store information in a database 211 or may communicate to a remote server. The CPU 201 may be configured to provide analysis or it may simply pass data retrieved from the sensors to the remote determination server which returns information to the CPU 201 or may simply store it or analyse it remotely. Further, the CPU 201 may pass it to third party entities such as doctors, health providers or cloud information services.

Illustrated in the system architecture is also a display 212 in communication with the CPU 201. The display is illustrated more clearly in FIG. 1. There may be an LED screen or any type of TV screen. On top of the display may be placed a dielectric mirror surface layer to enable the user to view themselves. In another embodiment, the display may simply be a feed from the camera such that the user can see themselves in the display. Preferably, the dielectric mirror surface enables the user to have a clear reflection of themselves in a manner they typically expect when viewing themselves in a mirror. A combination of dielectric mirror and LED screen enables the user to be presented with an image of themselves as well as the overlay provided by the CPU on the LED screen. A dielectric mirror or bragg mirror is a type of mirror composed of multiple thin layers of dielectric material typically placed on a substrate of glass or other optic material. It would of course be understood that although a dielectric surface mirror surface layer is preferable, any suitably reflective surface may be used.

Methods and processes described herein can be embodied as code (e.g., software code) and/or data. Such code and data can be stored on one or more computer-readable media, which may include any device or medium that can store code and/or data for use by a computer system. When a computer system reads and executes the code and/or data stored on a computer-readable medium, the computer system performs the methods and processes embodied as data structures and code stored within the computer-readable storage medium. In certain embodiments, one or more of the steps of the methods and processes described herein can be performed by a processor (e.g., a processor of a computer system or data storage system). It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that is capable of storing computer-readable information/data. Computer-readable media should not be construed or interpreted to include any propagating signals. The computer program may include a suitable operating system upon which the program to enable the present functionality may be executed.

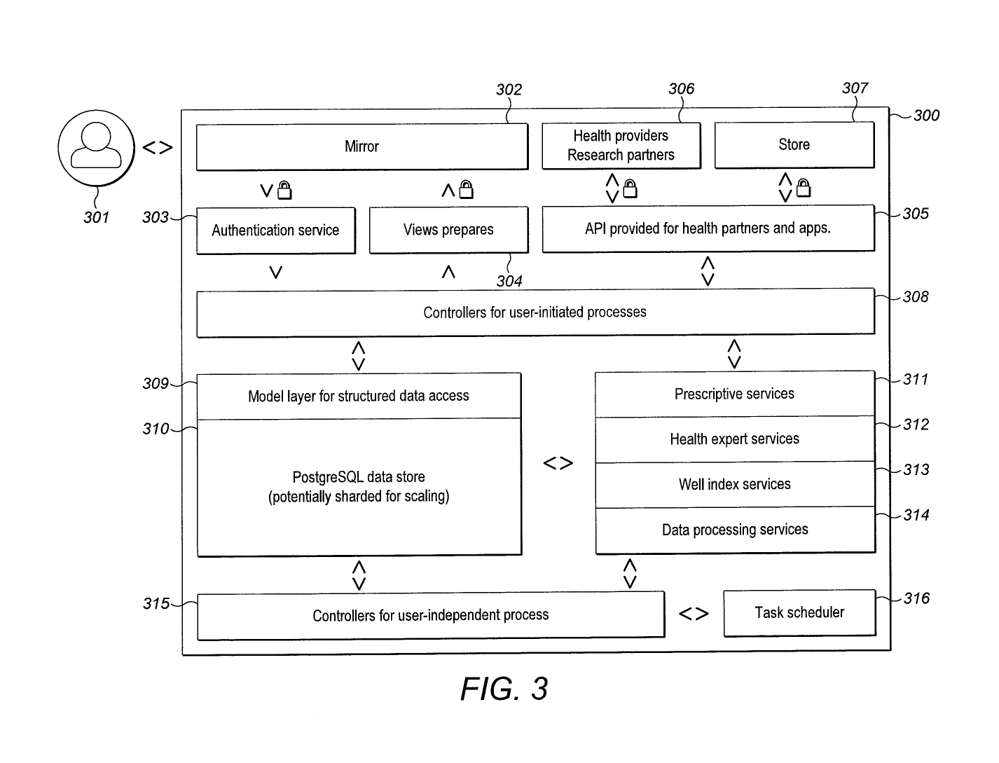

Above has been described a mirror based system which enables determination of biological information of a subject while they stand in front of the mirror. Optionally the passive measurement is presented to the user or passed or stored for subsequent diagnosis. It is proposed that the mirror based system includes an intelligent virtual data platform which is built around a set of algorithms which analyse vital signs and other data captured from a combination of sensors, motion detectors, and a video camera. The system enables real-time intelligent applications that can serve patients, consumers, providers, pharma companies, administrators, and policy makers. Such a system architecture is illustrated in FIG. 3. The platform illustrated in FIG. 3 is proposed to enable the interaction of multiple external sources as well as the interaction with the end user. The system architecture 300 is illustrated with a subject 301 positioned in front of the mirror system 302.

In this example, the mirror system 302 provides information to health providers and research partners 306. In some examples the mirror system comprises a CPU that is able to run a series of apps, each providing particular functionality such as diagnosis functionality or user interface functionality. For example, an email app might be configured to detect the user in front of the mirror and provide notifications of incoming emails. To facilitate downloading of such apps, the architecture may include a store 307 with which the mirror may be in communication. The store may be an application repository from which the user can download applications to be installed on the mirror system.

An open API 305 allows third party hardware vendors and specialised health care program apps to integrate with the system. Algorithms allow the system to measure and track other vitals, link up to electronic medical records, facilitate telemedicine with healthcare professionals, and provide personalised recommendations on customised programs for specific conditions such as chronic diseases, fertility, and early warning detection and alerts of certain health conditions.

The components are structured using an MVC-architecture with an additional component added for services. This will account for the added complexity when deploying machine learning models and data refining processes. The architecture can be broken down into the model, the service, the controller and the view.

The model comprises the abstraction model to the data layer. All input readings are preserved for verification and the model applies all required transformation steps on top of it. Some examples for Model classes are: readings from the devices and sensors, imported readings from wearables or other user- and health events.

Services provide pre-defined computations to other components, like Controller classes 308, 315. They may use input from Controllers and Models to run calculations, like data preparation, aggregation, classification and predictions. Services are where most research and enhancement will take place over time. Eventually our Controllers will run Service pipelines to calculate diagnostic and predictive results for users. For example, an Aggregation Service class gathers a user's wellness data from mirror- and wearable readings to combine it in a unified time series of observations. Upon completion, this step feeds into an Autocorrelation Service to identify unusual patterns or a regression-based prediction Service to give the risk of a future health condition.

Controller classes run a set of actions using Models and Services to potentially output views. They may be initiated by user actions or actions scheduled using time intervals. Examples could be regular data imports from other services, user action on the mirror or batch runs to handle computationally long-running processes.

To keep the software on the platform simple and robust, the backend will pre-compile all output data shown to the user on the mirror. This is achieved using Views on results or data. Examples for views are the display of time series, statistics or recommendations on the mirror. By treating Views as separate class entities, we will achieve a clean separation between presentation and the data layer with its subsequent computations applied.

The proposed technology stack supports the vision of scalable health. It gives users, data scientists, health care providers, pharma, payers, and Store app providers a common platform to share knowledge and data. To support our design goals of scalability, openness and security, we are using open source software wherever possible. While saving cost in the long run, this will also make our processes trustworthy and transparent.

The Data/Model layer will be powered by Postgresql 310, an advanced open source database known for stability and reliability. It also provides granular access controls to protect user data at every step. At the same time it can scale horizontally to help the system with seamless user growth.

An example programming language that can be used is Python 3 for its flexibility and wide availability of high-level libraries. Python libraries can be used to provide flexible API interfaces to hardware devices and connect to third-party services to synchronize additional data on a regular basis.

Libraries such as the Pandas and Numpy libraries may form the backbone of the data processing layer. They allow developers to focus on high-level tasks by handling the underlying details.

Python also features a large collection of machine learning and neural network libraries. Health experts and data scientists are able to use the same code during development and production, thus avoiding a lengthy porting process into a production language, like Java.

Performance-critical parts can still be outsourced to C-level libraries where needed.

Since the architecture is structured around Services, supported by a Model and Controller layer, the platform is able to constantly improve and experiment on our overall system without downtime. In-house health experts can easily add new Services and deploy them when ready. Logically separate Services will be isolated in independent virtualization environments, like Docker. This allows the platform to scale the number of Service instances as needed, while at the same time isolating Services from user data. It even allows for third party providers to develop and offer Services.

Security and privacy is built into the platform. To protect user's data, all data is anonymized. All health data will use an encrypted primary key. It can only be connected to a username by decrypting it with the user's password.

This approach also fulfils and exceeds the safeguard and policy recommendations stated in the Health Insurance Portability and Accountability Act (HIPAA).

The architecture allows for quick development of better usage of health data, while saving users the technical complexities and details. New data sources, insights and machine learning models can be quickly implemented. The exemplary use of Python as main programming language and modular architecture allows for fast development while staying flexible for the future.

Returning to FIG. 3, illustrated is an Authentication Service 303 that allows access to the smart mirror system by controllers for user-initiated processes 308. The controllers also provide views prepared for the smart mirror system 304. APIs are provided for health partners and apps which are also associated with the controllers 308.

A Model layer for structured data access 309 is provided associated with the PostgreSQL data store 310 which is potentially sharded for scaling. Data access to the store can be allowed from prescriptive services 311, health expert services 312, well index services 313 and data processing services 314 to enable various use cases described above. Controllers for user-independent processes 315 can also be provided associated with the services 311-314, the data store 310 and a task scheduler 316.

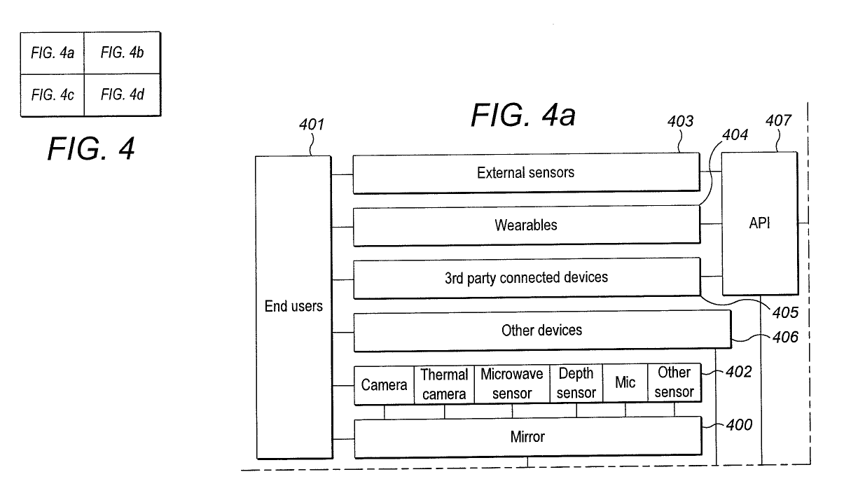

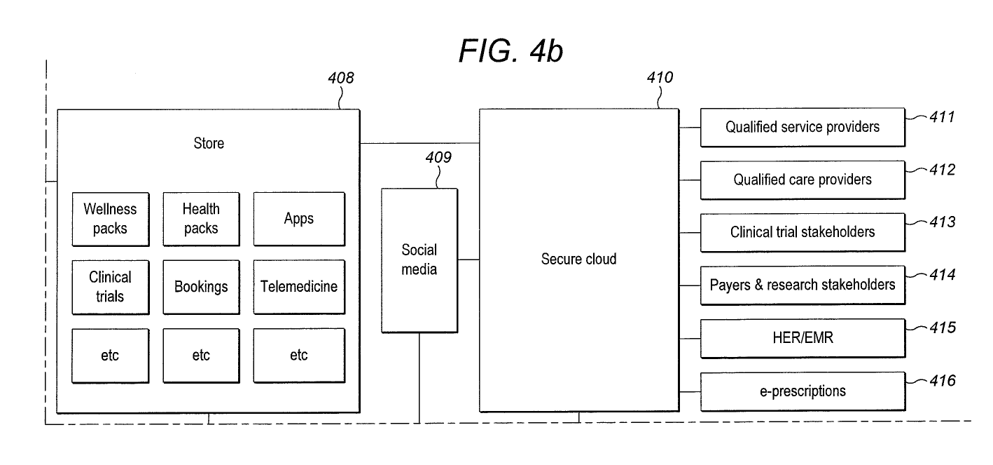

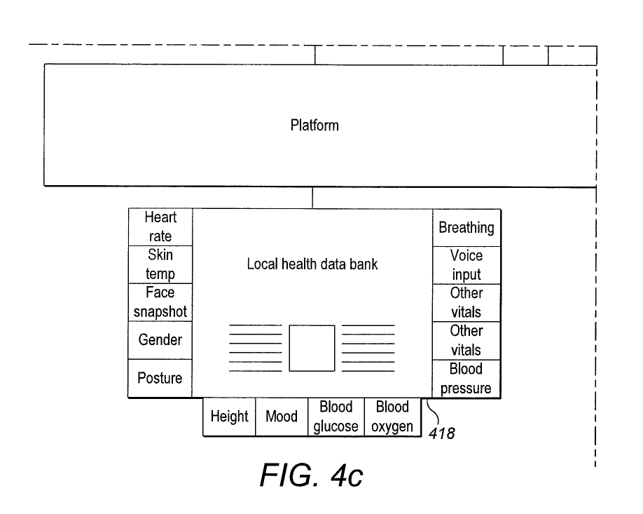

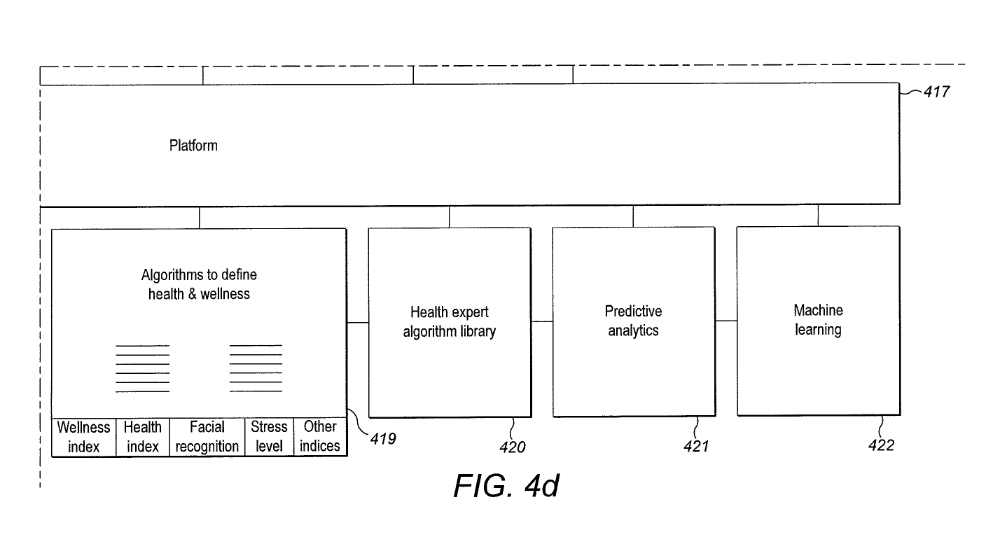

It is considered that the mirror system may be incorporated into wider system architecture. A example wider architecture is illustrated in FIG. 4. In this figure, the mirror system 400 is illustrated within the context of a wider healthcare ecosystem that enables interaction between the system and external access by the end user 401. Access to the system is provided by the mirror system described above and illustrated here as item 400 or via a web interface to the platform 417 where the user is able to access their data. A platform 417 is provided in this example. The platform 417 is the brains that connects all the stakeholders together and provides personalized and disseminated information at the right place and time to the right stakeholder. Through the use of a central platform 417 in this way, the system may be scalable, versatile and flexible to allow access by the end user and multiple stakeholders or components. The platform may be modular to provide additional functionality.

The exemplary wider architecture of FIG. 4 will now be described with each component typically interacting with the central platform 417. End users 401 interact with the platform 417 through the mirror system 400 and other connected devices 406. The primary interface/hub would be through the mirror system 400. Sensors 402 are used to determine biological information from the users and also to provide and receive instructions or information. Sensors 402 can be used in combination with each other to error correct or improve reliability of measurements, for example the visual feed could also be used to calibrate/compensate other measurements if they're affected by the mirror not being hung perfectly level.

External sensors 403 such as force and pressure sensors, as well as sensors around the home that could detect falls for example but also medical devices such as blood glucose devices, pressure monitors, DNA, among others, could be used depending on the clinical use of the system. Furthermore, this can be applied to wearables 404 such as pedometers for example. The sensors 403, wearables 404 or other connected devices 405 can connect directly to the platform 417 or via an API 407.

Additionally, data can be collected from connected sources. This includes validating, normalizing and cleaning the incoming data. The system is operable to group, refine and aggregate a user's health data to present it on the device, in this example, the mirror 302. The system is thus able to provide the user with relevant time series, historical comparisons and health indicators.

The platform may be connected to a local database or local health data bank 418. In one embodiment this is a data storage location of all of a user's health data such that data is stored in one place and not in several remote locations as is currently the case.

An app store 408 may also be provided. The app store 408 or application repository is a place for end users to download different apps that focus on certain use cases. Examples include weight loss/diabetes management/booking appointments with care providers/etc. Apps can be in the form of widgets that download and link up to other aspects of the platform, e.g. weather, calendar together with your mood and health goals to suggest a good time to go for a jog that is a free slot in the calendar and the weather is good, among others.

A social media aspect 409 may also be provided which may allow users to share their health data after giving permission. This can allow people to monitor their loved ones remotely as well as community groups for people with similar conditions.

Further, users may wish to store their health data remotely. For this purpose, a secure cloud 410 is illustrated which is an encrypted and anonymized data storage for health data/algorithms/platform/etc.

The secure cloud also allows for access to be given to remote authenticated stakeholders. For example, qualified service providers 411 may provide services such as therapies and other health, wellness, and lifestyle improvement services. This can be done through the platform connecting them via the mirror system interface. Only required data will be disseminated to each provider based on what information is required to carry out the service as well as permission given by the end user. In some cases, data maybe anonymized and then through the secure cloud 410 transmitted back to the correct end user.

Optionally, qualified care providers 412 may be granted access but these are more closely related to medical and health care provision and services. A care team can be used to best interact with a customized care plan for each patient (end-user).

Further, Clinical Trial stakeholders 413 may be given access. Many clinical trials are contemplated, whether for the first time generating algorithms for objectively measuring pain, mental health, or other just for data validation and obtaining new data sets that typically would not be available to pharma companies during clinical trials.

Research stakeholders 414 may also be granted access for academic/medical research as well as insurance companies to help better manage their higher risk patients with multiple chronic conditions

Further, the secure cloud 410 may link to each patient's electronic health record and electronic medical records 415. This in combination with the platform's health bank 418 will allow for a complete picture of each user and allowing both the user to take control of their health and the care team to provide a more tailored care plan. This information on a bigger scale will allow the system to better understand trends, predictive analytics on both a personal as well as a population health standing.

Optionally the data may be provided to an e-prescriptions service 416. Medication information may be stored that will allow the platform to keep track of and make reminders on medication prescription etc. Combined with frequent measurement of vitals, it will also allow the system to monitor how the drugs prescribed and taken are having an effect on people with similar conditions, but different variables in health etc.

The platform 417 may utilise a series of algorithms to define health and wellness 419. Combinations of different vitals being measured and recorded from the mirror 400 and other devices, stored in 418, may allow different algorithms to be created to generate health and wellness indices to establish how well and health the user is. For example, a combination of heart rate, mood, voice tonality, posture, and facial prompts will allow the system to measure and learn over time stress levels of each individual. The algorithms may be provided in a remote module in communication with the platform 417 or may be provided within the module itself. Alternatively, the platform may pass the algorithm information to the mirror, devices or apps from the store.

Further, algorithms may be provided from external sources such as health experts and stored in a library 420. Researchers may be able to program algorithms directly into the platform and use its health bank and other machine learning capacity to identify and monitor certain conditions. For example, studies show that monitoring retina can potentially detect risk of type 2 diabetes or observing the colour of skin can detect whether or not a new transplant patient is accepting or rejecting a new organ. In a further example, by using the visual feed, and the audio, combined changes in facial symmetry and slurring of words and weakness in balance to detect for stroke, and trigger the style of questions used to quickly triage this. A similar scenario using the optics and the microwave sensor could determine a heart attack, for example heart fibrillation and left-side motor control issues could trigger triage questioning.

Predictive analytics 421 and machine learning 422 modules may allow the platform increased functionality. The module may provide the user with predictive analytics mined from her/his own health data and simple questions asked by the platform in an unobtrusive way. The system may also combine proven research findings with predictive machine learning (ML) techniques, like regression and classification, Neural Networks and Support Vector Machines. By bringing together medical research and well-established ML techniques, the system may be able to predict the risk of future disease or provide physicians with warnings on possible relapses after treatments.

The present description provides a seamless and scalable ecosystem that tracks the body's vitals, mood, and performance and provides targeted recommendations through products we use every day in our lives. The system provides contactless passive measurement of your vitals every time you stand in front of mirror. Additionally, a seamless intelligent virtual data platform is proposed built around a set of algorithms which analyse vital signs and other data.

Although the present invention has been described in connection with specific exemplary embodiments, it should be understood that various changes, substitutions, and alterations apparent to those skilled in the art can be made to the disclosed embodiments without departing from the spirit and scope of the invention as set forth in the appended claims.

1. A system for determining biological information of a subject, comprising:
a surface configured to display an image of a subject positioned in front of the surface;
a sensor configured to identify data of the subject;
an analysis and control unit configured to analyse the data and determine biological information of the subject based on the data.

2. The system of claim 1, wherein the surface comprises a mirrored surface.

3. The system of claim 1, wherein the surface comprises a display.

4. The system of claim 1, wherein the system further comprises a digital platform, wherein the platform provides a series of application programing interfaces “APIs”, each API providing access for third party components to the data or the biological information or both.

5. The system of claim 2, wherein the mirrored surface is a dielectric mirror surface arranged adjacent to a display.

6. The system of claim 1, wherein the sensor is a thermal infrared “IR” camera.

7. The system of claim 6, wherein the analysis unit is configured to determine skin temperature based on data from the thermal IR camera.

8. The system of claim 1, wherein the sensor is a microwave pulse sensor configured to omit a signal and receive reflected signals, the reflected signals signal including at least part of the omitted signal reflected off the subject.

9. The system of claim 1, wherein the system further comprises an ambient temperature sensor.

10. The system of claim 1, wherein the sensor is a microphone.

11. The system of claim 1, wherein the sensor is a camera.

12. The system of claim 1, wherein the sensor is a 3D depth sensor apparatus.

13. The system of claim 1, wherein the system further comprises one or more force sensors.

14. The system of claim 1, wherein the biological information is any one or more of mood, age, gender and/or stress.

15. The system of claim 1, wherein the system comprises a plurality of different sensors and wherein the biological information is determined based on a combination of the data retrieved from each of the sensors.

16. The system of claim 1, wherein the system further comprises a digital platform.

17. The system of claim 1, wherein the system comprises an algorithm library comprising a series of algorithms for the analysis unit to use when determining biological information from the data.

18. The system of claim 1, wherein the system comprises a module which enables predictive analytics of future conditions of the subject based on the biological information.

19. A method in a system comprising a mirrored surface, a display and one or more sensors configured to identify data of a subject positioned in front of the mirrored surface, the method comprising:
retrieving data from the one or more sensors;
identifying a biological information of the subject based on the data; and,
outputting the biological information to the display.

20. A computer readable medium comprising instructions which when executed by a computer cause the computer to:
retrieve data from one or more sensors;
identify biological information of a subject positioned in front of a mirrored surface adjacent to a display based on the data; and,
outputting the biological information to the display.
